(19) United States
(12) Patent Application Publication
(10) Pub. No.: US 2026/0071957 A1
(43) Pub. Date: Mar. 12, 2026

(54) METHOD FOR DETECTING TARGET ANALYTE USING HYDROGEL AND BIO-SENSING DEVICE UTILIZING SAME

(71) Applicants: KOREA INSTITUTE OF MACHINERY & MATERIALS, Daejeon (KR); INDUSTRY-ACADEMIC COOPERATION FOUNDATION, YONSEI UNIVERSITY, Seoul (KR)

(72) Inventors: Gee Hong KIM, Daejeon (KR); Jae-Jong LEE, Daejeon (KR); Hyung Jun LIM, Daejeon (KR); Won-Gun KOH, Seoul (KR); Se Min KIM, Seoul (KR)

(21) Appl. No.: 18/872,927

(22) PCT Filed: Jun. 10, 2022
(86) PCT No.: PCT/KR2022/008261

§ 371 Date: Dec. 9, 2024

(51) Int. Cl. G01N 21/45; G01N 21/75; G01N 33/549

(52) U.S. Cl. CPC G01N 21/45; G01N 21/75; G01N 33/549

(57) ABSTRACT

A method for detecting target analyte using hydrogel and biosensing device using the method are presented. In the method, first and second polymer hydrogels having first and second patterns respectively are prepared. The first polymer hydrogel has a target analyte-specific probe coupled thereto and has target analyte sensitivity. A target analyte is contacted with the first polymer hydrogel to induce a volume change of the hydrogel. A change in a moiré pattern generated by overlapping the first and second polymer hydrogels with each other is obtained from an image monitoring device.

### BACKGROUND

### 1. Field of Disclosure

The present disclosure of invention relates to a method for detecting target analyte using hydrogel and biosensing device using the same.

### 2. Description of Related Technology

Hydrogels are hydrophilic polymers with a three-dimensional structure that can easily contain a large amount of water and genes, proteins, and cells. Because the hydrogels have properties similar to living tissue, the hydrogels have high biocompatibility and have been widely used in biomedical fields such as artificial organs, biosensors, drug delivery systems, cosmetics, and tissue engineering for a long time.

In addition, in cases that the hydrogels undergo structural or chemical changes in the polymer chains that compose the hydrogel or change in the degree of cross-linking of the polymer chains due to external control factors such as temperature, pH, and ionic strength, the chemical energy balance formed between the hydrogel and water molecules changes, and thus water molecules flow in or out, causing the volume of the hydrogels to expand or decrease.

The hydrogel whose physical properties, such as volume, degree of cross-linking, and strength, change as its structure changes in response to the external environment is called a dynamic hydrogel. These dynamic hydrogels are actively utilized in biotechnology fields such as research on changing the physiological behavior of cells and tissues, tissue engineering, drug delivery, and Bio-MEMS.

The dynamic hydrogels are manufactured by mixing and stirring monomers containing organic molecules, proteins, etc., and then crosslinking and curing them by ultraviolet irradiation or heating. As a representative example of using the dynamic hydrogels as polymer materials for drug delivery, there are studies on systems in which phenylboronic acid is used to react with glucose, causing the hydrogels containing phenylboronic acid to swell and release drugs or genes loaded inside. Among them, Korean Patent No. 10-2075476 discloses a hydrogel including a polymer combined with a natural product having a cis-diol functional group and phenylboronic acid, and a drug delivery system using the same. Even if it is the hydrogel that is highly responsive to external stimuli, such as glucose, pH, and active oxygen, in reality, it is difficult to manufacture the hydrogels with a volume shrinkage ratio of less than 70%, which limits the active use of dynamic hydrogels as biosensors.

Related prior art is Korean Patent No. 10-2075476 (Feb. 4, 2020).

### SUMMARY

The present invention is developed to solve the above-mentioned problems of the related arts. The present invention provides a method for detecting a target analyte using a hydrogel, wherein the volume change rate of the hydrogel is quantified through a moiré signal, and the presence or absence of a target analyte and quantitative detection may be easily implemented.

In addition, the present invention also provides a method for detecting a target analyte that does not require a separate labeling substance, as it detects the target analyte through moiré pattern analysis, unlike conventional optical analysis methods that use fluorescent substances, by utilizing the transparent properties of the hydrogel.

In addition, the present invention also provides a biosensing device using the method for detecting a target analyte that may detects various types of markers without limitation by involving a monomer with a desired biochemical functional group in the polymerization process of a polymer chain,

According to an example embodiment, in the method, first and second polymer hydrogels having first and second patterns respectively are prepared. The first polymer hydrogel has a target analyte-specific probe coupled thereto and has target analyte sensitivity. A target analyte is contacted with the first polymer hydrogel to induce a volume change of the hydrogel. A change in a moiré pattern generated by overlapping the first and second polymer hydrogels with each other is obtained from an image monitoring device.

In an example, the target analyte-specific probe may be cross-linked with a polymer chain inside the first polymer hydrogel and is fixed to a hydrogel surface.

In an example, the second pattern may be a reference pattern that overlaps the first pattern to form the moiré pattern.

In an example, the method may further include quantifying a change in the moiré pattern.

In an example, the change of the moiré pattern may be induced by a probe immobilized in a polymer hydrogel sensitive to the target analyte and a shrinkage of the hydrogel by a specific binding of the target analyte.

In an example, each of the first and second patterns may be in a form of a parallel grid with a regular interval of 5 nm to 100 nm.

In an example, the probe may be at least one selected from a group consisting of an aptamer, a peptide, an enzyme, a hormone receptor, an antibody, an antigen, and a cell.

In an example, a polymer may be at least one selected from a group consisting of polyethylene glycol (PEG), polyvinyl alcohol (PVA), polyvinyl pyrrolidone (PVP), polyacrylamide, polyacrylic acid and copolymers thereof, alginate, agarose, cellulose, gelatin, collagen, hyaluronic acid and chitosan.

In an example, the target analyte-specific prove may have an acrylate functional group introduced.

According to an example embodiment, the biosensing device includes a reaction part 30, a light source part 10, a reference pattern part 20, an imaging part 40 and a processing part 50. The reaction part 30 includes a first polymer hydrogel having a first pattern. The first polymer hydrogel has target analyte-specific probe coupled thereto and having target analyte sensitivity. The light source part 10 provides a light to the reaction part 30. The reference pattern part 20 includes a second polymer hydrogel having a second pattern, and disposed in an optical path between the reaction part 30 and the light source part 10. The second pattern is projected onto the first pattern by the light provided by the light source part 10 to form a moiré pattern. The imaging part 40 is configured to make an image of the first pattern and the second pattern projected onto the first pattern. The processing part 50 is configured to analyze target analyte information based on the moiré pattern obtained by the imaging part 40. The moiré pattern changes as the target analyte binds to the target analyte-specific probe.

In an example, the target analyte-specific probe may be cross-linked with a polymer chain inside the first polymer hydrogel and fixed to a hydrogel surface.

In an example, the second pattern may be a reference pattern that overlaps the first pattern to form the moiré pattern.

In an example, a change of the moiré pattern may be induced by a probe immobilized in a polymer hydrogel sensitive to the target analyte and a shrinkage of the hydrogel by a specific binding of the target analyte.

In an example, the probe may be at least one selected from a group consisting of an aptamer, a peptide, an enzyme, a hormone receptor, an antibody, an antigen, and a cell.

In an example, a polymer may be at least one selected from a group consisting of polyethylene glycol (PEG), polyvinyl alcohol (PVA), polyvinyl pyrrolidone (PVP), polyacrylamide, polyacrylic acid and copolymers thereof, alginate, agarose, cellulose, gelatin, collagen, hyaluronic acid and chitosan.

According to the present example embodiments, by quantifying the volume change rate of the hydrogel through the Moiré signal, the presence and quantitative detection of target analyte may be easily implemented.

In addition, by utilizing the transparent properties of the hydrogel, the target analyte is detected through moiré pattern analysis, unlike conventional optical analysis methods that utilize fluorescent substances, so there is no need to add labeling substances, and detection may be performed in a simple manner.

In addition, by involving compounds with biochemical functional groups in the polymer chain polymerization process, it provides the advantage of being able to detect various types of markers.

In addition, by amplifying the detection signal of the target analyte through changes in the moiré pattern, detection of extremely small amounts of the target analyte is possible, and the amplification effect of the detection signal may be easily controlled by controlling the concentration of the target analyte-specific probe.

### BRIEF DESCRIPTION OF THE DRAWINGS

FIG. 1A and FIG. 1B are schematic diagrams showing volume change and moiré pattern change due to external stimulation of hydrogel according to an example method for detecting target analyte using hydrogel of the present invention;

FIG. 2 is a schematic diagram showing the volume shrinkage mechanism of hydrogel according to binding of a target analyte probe and target analyte in patterned hydrogel according to the example method;

FIG. 3 is a schematic diagram showing a process of the example method;

FIG. 4 is a graph showing results of measuring the selectivity of hydrogel according to the example method, a first comparative example and a second comparative example;

FIG. 5 is a graph showing a change in hydrogel volume according to the amount of antibody inserted into the hydrogel manufactured by the example method;

FIG. 6 is a graph showing a change in moiré pitch size according to BDNF protein concentration;

FIG. is a graph comparing a change in pitch size of the hydrogel and moiré manufactured by the example method; and

FIG. 8 is a block diagram showing a biosensing device using the method.

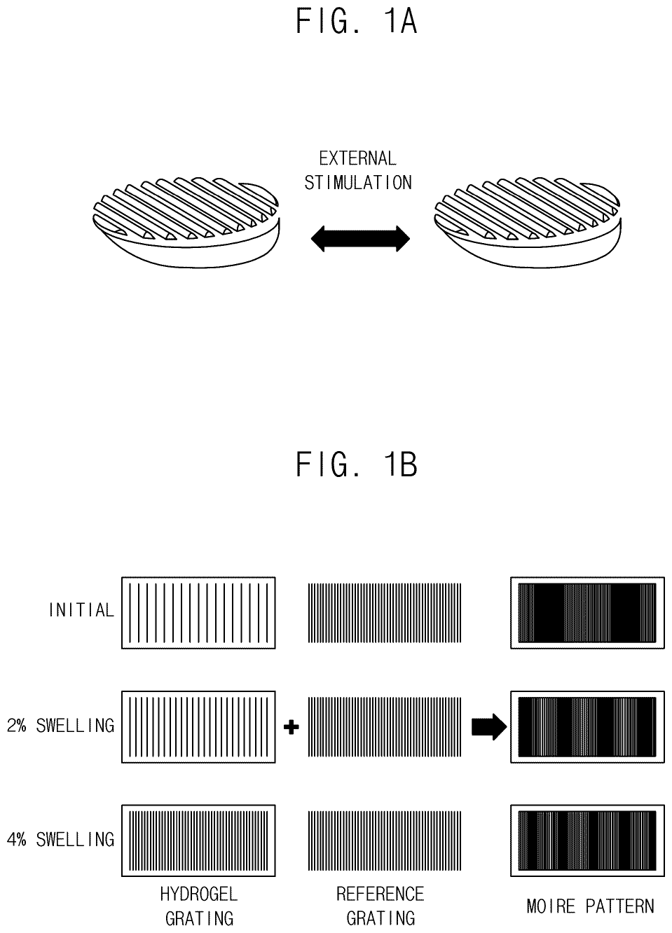

### DETAILED DESCRIPTION

Hereinafter, a method for detecting a target analyte using a hydrogel according to the present invention and a biosensing device using the same will be described in detail. In this case, unless otherwise defined, all technical and scientific terms have meanings commonly understood by those of ordinary skill in the art to which this invention belongs, and the terms used in the description of the present invention are only intended to effectively describe specific embodiments and are not intended to limit the present invention.

In addition, in the following description, descriptions of well-known effects and configurations that may unnecessarily obscure the gist of the present invention are omitted. In the following specification, units used without special mention are based on weight, and for example, a unit of % or ratio means weight % or weight ratio.

In addition, when describing components of the present invention, terms such as first, second, A, B, (a), (b), etc. may be used. These terms are only intended to distinguish the components from other components, and the nature, order, or sequence of the components are not limited by these terms.

Additionally, the singular forms used in the specification of the present invention may be intended to include the plural forms as well, unless the context specifically indicates otherwise.

Hereinafter, a method for detecting a target analyte using a hydrogel according to the present invention will be described in detail.

In the method according to the present example embodiment, first and second polymer hydrogels having first and second patterns respectively are prepared. The first polymer hydrogel has a target analyte-specific probe coupled thereto and has target analyte sensitivity. A target analyte is contacted with the first polymer hydrogel to induce a volume change of the hydrogel. A change in a moiré pattern generated by overlapping the first and second polymer hydrogels with each other is obtained from an image monitoring device.

The hydrogel is a cross-linked network polymer composed of one or more monomers, and has high water content, so various biomolecules may be immobilized within the hydrogel while maintaining their structure and activity. The present example embodiment relates to a method for immobilizing the target analyte-specific probe on the hydrogel and detecting the target analyte through a change in the volume of the hydrogel according to binding to the target analyte. Furthermore, the present example embodiment enables quantitative detection of the target analyte with high sensitivity by amplifying the change in volume of the hydrogel through a moiré signal.

In the present example embodiment, the target analyte-sensitive first polymer hydrogel including the first pattern may be one in which the target analyte-specific probe is immobilized on the surface of the hydrogel by being cross-linked with the polymer chain inside the first polymer hydrogel.

The target analyte-specific probe is a biomolecule that recognizes the analyte, and may be at least one selected from the group consisting of aptamers, peptides, enzymes, hormone receptors, antibodies, antigens, and cells that are capable of selectively reacting and binding with the target analyte.

The target analyte-specific probe may have a predetermined functional group introduced thereto in order to be fixed to the hydrogel, and in one embodiment, the target analyte-specific probe may have an acrylate functional group introduced thereto.

As the target analyte-specific probe is incorporated into the polymer chain forming the hydrogel, the target analyte-specific probe may be sensitive to the target analyte, and the target analyte-specific probe and the target analyte may form a bond. To induce a volume change of the hydrogel upon binding of the target analyte, one target analyte molecule may interact with two or more target analyte-specific probes. Accordingly, the target analytes combined with two or more target analyte-specific probes may induce volume shrinkage of the hydrogel by forming physical cross-linking points.

The first polymer may be the same as or different from the second polymer, and the polymer is not limited as long as the linear polymer constituting the hydrogel is a polymer that is water-soluble. For example, the polymer is at least one selected from a group consisting of polyethylene glycol (PEG), polyvinyl alcohol (PVA), polyvinyl pyrrolidone (PVP), polyacrylamide, polyacrylic acid and copolymers thereof, alginate, agarose, cellulose, gelatin, collagen, hyaluronic acid and chitosan. The polymer may be a synthetic polymer or a natural polymer capable of fixing a biomolecule, and is not particularly limited thereto.

Each of the first pattern and the second pattern may be a fishbone pattern, a ladder pattern or a parallel grid pattern. Specifically, the parallel grid pattern refers to a pattern in which multiple parallel straight lines of a certain thickness are arranged at regular intervals. Preferably, the pattern may be formed at a constant interval of 5 to 100 nm, more preferably, it may be formed at an interval of 10 to 80 nm, and most preferably, it may be formed at an interval of 15 to 60 nm. The line width in the pattern may be 0.5 to 50 nm, preferably 1 to 20 nm, but is not particularly limited thereto. The first and second patterns are manufactured to exhibit similar line widths and spacings, which makes it easy to measure changes in the moiré pattern.

The step of preparing the target analyte-sensitive first polymer hydrogel including the first pattern coupled with the target analyte-specific probe according to the present example embodiment is specifically as follows.

For example, a biomolecule having an acrylate functional group introduced thereto is manufactured by treating a biomolecule such as an antibody capable of forming a specific binding with a target analyte with N-hydroxysuccinimide acrylic acid. The biomolecule with an acrylate functional group is mixed into a polymer precursor solution, a crosslinking agent, an initiator, and a catalyst are added, and then a hydrogel is prepared through UV photopolymerization. At this time, when a polymerization solution containing a precursor solution, a crosslinking agent, an initiator, and a catalyst for polymerization is added to a mold having a certain pattern and polymerized, the hydrogel having a desired pattern may be obtained. Since the patterning method may be performed using conventional techniques known in the art, a detailed description is omitted.

The polymer precursor solution may contain a porogen. The pore inducer may be, for example, an inorganic oxide including silica, titania, zirconia, a derivative thereof, or a mixture thereof. The pore-inducing agent is not cross-linked during the polymerization process and is subsequently removed, thereby forming pores within the hydrogel, thereby obtaining a porous hydrogel.

The hydrogel may be further coated with platinum. Platinum may be coated on the hydrogel to a thickness of 10 nm to 50 nm, preferably 20 nm to 30 nm, thereby controlling the refractive index of the hydrogel surface to obtain a clear pattern image. Since excessive coating may darken the image, it is possible to easily measure the moiré signal by coating with a thickness within the above-described range.

The second polymer hydrogel including a second pattern may be manufactured in the same manner as described above. However, when used for the purpose of forming a moiré pattern by projecting onto the first pattern, it is not necessary for the target analyte-specific probe to be immobilized.

In general, the moiré pattern refers to an interference fringe created when two or more periodic patterns overlap. In academic terms, it can be defined as a unique low-frequency pattern that occurs due to the beat phenomenon when multiple gratings with similar periods overlap.

When a light is irradiated onto the second polymer hydrogel including the second pattern while a light source is placed, the shadow of the second pattern overlaps the surface of the first pattern, thereby forming the moiré pattern, which is defined as an initial moiré signal.

When a target analyte is brought into contact with the first polymer hydrogel, a specific binding is formed between the target analyte-specific probe and the target analyte, which is immobilized by cross-linking with the polymer chains within the hydrogel. As the probe and analyte are combined, the hydrogel shrinks, and a slight pitch change occurs in the pattern arrangement according to the change in the volume of the hydrogel. Accordingly, the initial moiré signal changes, and through this change, the presence or absence of a target material may be simply determined, and quantitative detection is possible by analyzing the intensity of the moiré signal.

At this time, preferably, two or more probes fixed to the internal chains of the hydrogel along the pattern may form specific bonds with one target analyte, and the degree of shrinkage of the hydrogel may be increased to maximize the amplification of the moiré signal.

As shown in FIG. 7, it may be confirmed that the pitch size of the hydrogel decreases by about 2.5% as the probe fixed to the internal chain of the hydrogel forms a specific bond with the target analyte, while the pitch size of the moiré pattern decreases by about 9.6%, resulting in a signal amplification effect of about 4 times. This shows that the method according to the present example embodiment, which induces a moiré pattern change, is effective in detecting trace amounts of the target analyte.

Specifically, the limit of detection (LOD) of the sensing technology using the moiré signal according to the present example embodiment was at the nanomolar level (˜29.62 nM), and it was confirmed that it was very effective in detecting the target analyte due to high sensitivity and selectivity.

The biosensing device 100 includes a reaction part 30, a light source part 10, a reference pattern part 20, an imaging part 40 and a processing part 50. The reaction part 30 includes a first polymer hydrogel having a first pattern. The first polymer hydrogel has target analyte-specific probe coupled thereto and having target analyte sensitivity. The light source part 10 provides a light to the reaction part 30. The reference pattern part 20 includes a second polymer hydrogel having a second pattern, and disposed in an optical path between the reaction part 30 and the light source part 10. The second pattern is projected onto the first pattern by the light provided by the light source part 10 to form a moiré pattern. The imaging part 40 is configured to make an image of the first pattern and the second pattern projected onto the first pattern. The processing part 50 is configured to analyze target analyte information based on the moiré pattern obtained by the imaging part 40. The moiré pattern changes as the target analyte binds to the target analyte-specific probe.

The hydrogel on which the first pattern is formed and the hydrogel on which the second pattern is formed are spaced apart and overlap each other, and when light irradiated from the light source part 10 passes through the reference pattern part 20, a shadow is generated by the reference pattern unit, and an image is formed on the first pattern on the surface of the hydrogel due to this shadow. At this time, the moiré pattern is formed by the hydrogel in which the first pattern is formed and the pattern by the shadow of the reference pattern part 20.

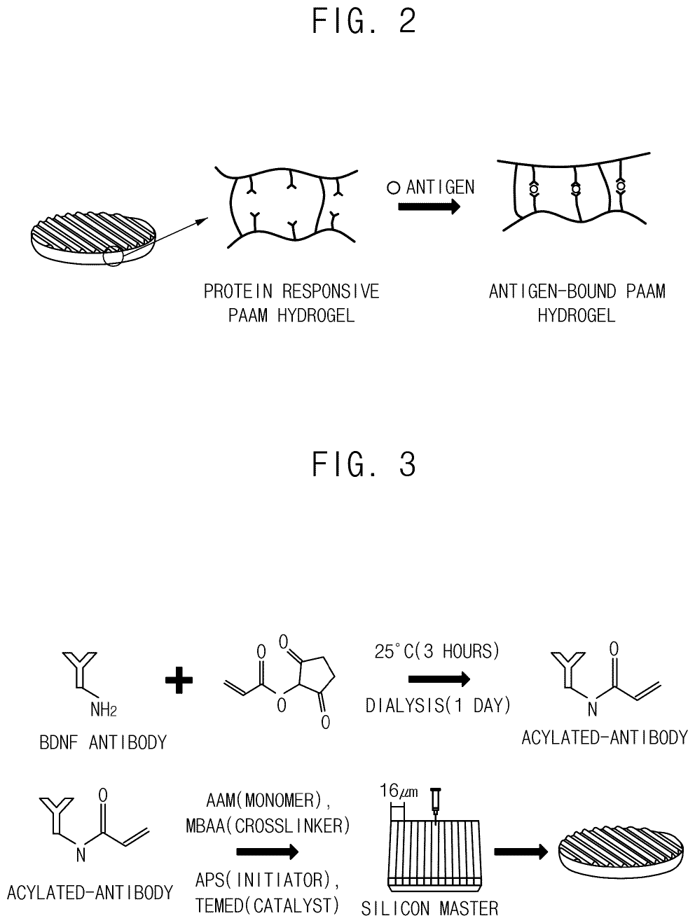

As illustrated in FIG. 2, when the reaction part 30 is provided with a target analyte, binding occurs with a target analyte-specific probe that is attached and cross-linked to the inside of the hydrogel, and volume shrinkage of the hydrogel in which a first pattern is formed by binding with the target analyte and its specific probe can be induced. Thus, the first pattern is changed. At this time, the moiré pattern generated by the second pattern being projected onto the first pattern by the light source irradiated from the light source part 10 is different from the moiré pattern before the target analyte is provided, and a change in the moiré pattern is observed when the target analyte is detected.

The change in the moiré pattern is captured by the imaging part 40, and the target analyte information is analyzed by the processing part 50 based on the captured image information. For example, by moving the straight lines in the vertical direction of the moiré pattern using an optical program, the waveform of the corresponding area may be checked. The waveform is decomposed into a single wave through Fourier transform to obtain intensity information for each wavelength.

According to the following [Equation 1], the moiré signal may be expressed as a function of the pitch size of the hydrogel and the pitch size of the reference pattern.

$$\frac{1}{{f}_{M}} = \vert \frac{1}{{f}_{H}} - \frac{1}{{f}_{R}} \vert \qquad [ Equation 1 ]$$

In the [Equation 1], f_{M} is the moiré pitch size, f_{H} is the hydrogel pitch size, and f_{R} is the pitch size of the reference pattern. According to the [Equation 1], as the hydrogel pitch size approaches the pitch size of the reference pattern, the moiré signal increases, and as the hydrogel pitch size increases, the moiré signal decreases. These moiré signals are decomposed into single waves through Fourier transform and programmed, and quantitative analysis may be performed by analyzing the intensity of the wavelength of each single wave.

The moiré signals quantify the volume change of the hydrogel, and offer the advantage of being able to easily detect even minute volume changes due to trace amounts of target analytes through signal amplification.

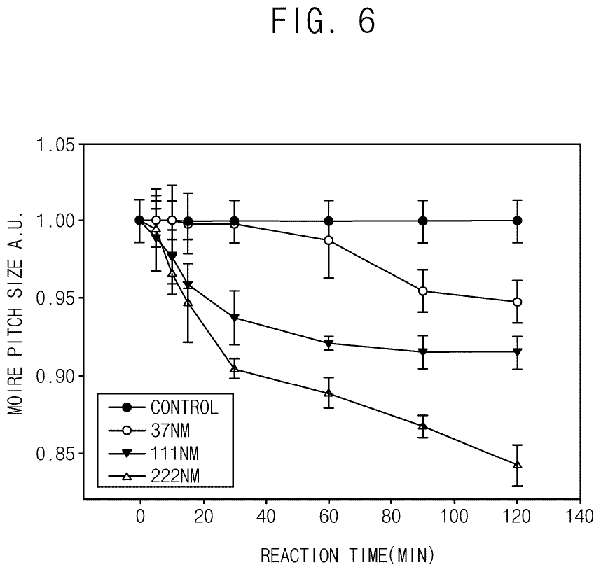

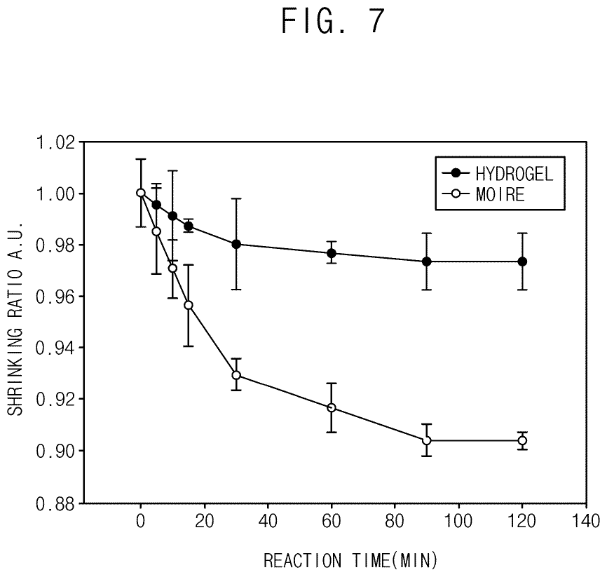

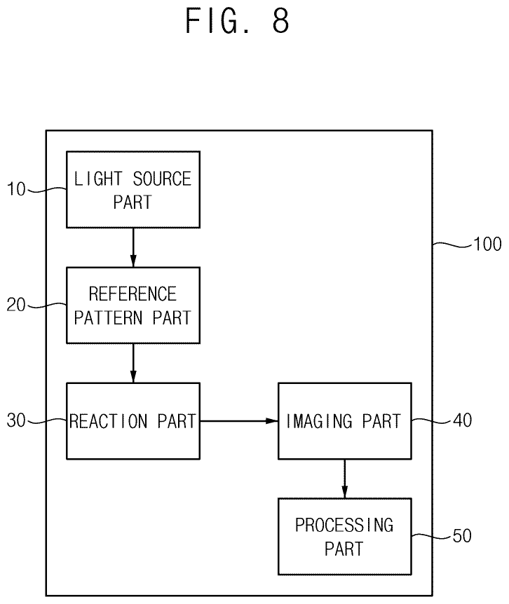

FIG. 6 is a graph showing the change in a moiré pitch size according to BDNF protein concentration. That is, FIG. 6 is a graph showing the decrease in the moiré pitch size according to the reaction time at different concentrations of BDNF, with the concentrations being 37 nM, 111 nM, and 222 nM.

Specifically, BDNF-sensitive hydrogels were added to 300 μL solutions containing 37 nM, 111 nM, and 222 nM BDNF at different concentrations, and reacted. Then, microscopic images of changes in the hydrogel and the moiré signals over time (0 min, 30 min, and 120 min) were captured, and the moiré signals of the same area were measured and compared and analyzed from the computer images using an optical program.

In addition, after drawing a line in the vertical direction of the moiré signal in the above video image, the brightness of the line profile area was analyzed and converted into the intensity of the black/white signal.

Thus, as shown in FIG. 6, the decrease in the moiré pitch size according to the reaction time at different concentrations of BDNF was shown. The moiré pitch size showed the greatest decrease rate at 222 nM, and a tendency to decrease depending on the concentration was observed.

Hereinafter, the method for detecting the target analyte using the hydrogel according to the present invention will be described in more detail through examples. However, the following examples are only a reference for explaining the present invention in detail, and the present invention is not limited thereto and may be implemented in various forms.

### Manufacturing Example 1. Manufacturing of Hydrogel with First Pattern Formed

Brain-derived neurotrophic factor (BDNF)-sensitive polymer hydrogel was prepared as follows. Anti-BDNF was dissolved in 100 μL of PBS buffer to a concentration of 1 mg/mL, and 33.3 μL of 2.22 mmol anti-BDNF was reacted with acrylic acid-NHS at 25° C. for 3 hours to produce modified antibody vinyl-BDNF. At this time, the ratio of anti-BDNF and acrylic acid-NHS was 1:6. To remove unreacted substances, dialysis was performed for one day using a 2000 MWCO dialysis kit. After dialysis, 0.211 mmol acrylamide and 6.486 mol N′, N′-methylenebisacrylamide (MBAA) were dissolved in the modified antibody solution, and the volume was made 97 μL after adding PBS buffer solution. After adding 2.5 μL of 10 wt % ammonium persulfate (APS) and 0.5 μL of tetramethylethylenediamine (TEMED), mixing for 1 second, a pattern was formed on a silicon wafer with a line pattern having a pitch size of 16 μm using a 1 mm thick silicon mold. After covering with a cover glass, the hydrogel was polymerized at 25° C. to produce an anti-BDNF-immobilized hydrogel. The hydrogel thus produced was washed with purified water, completely dried, and coated with 20-30 nM of platinum for 250 s to complete the BDNF-sensitive hydrogel.

### <Example 1> Selectivity Measurement for BDNF Detection

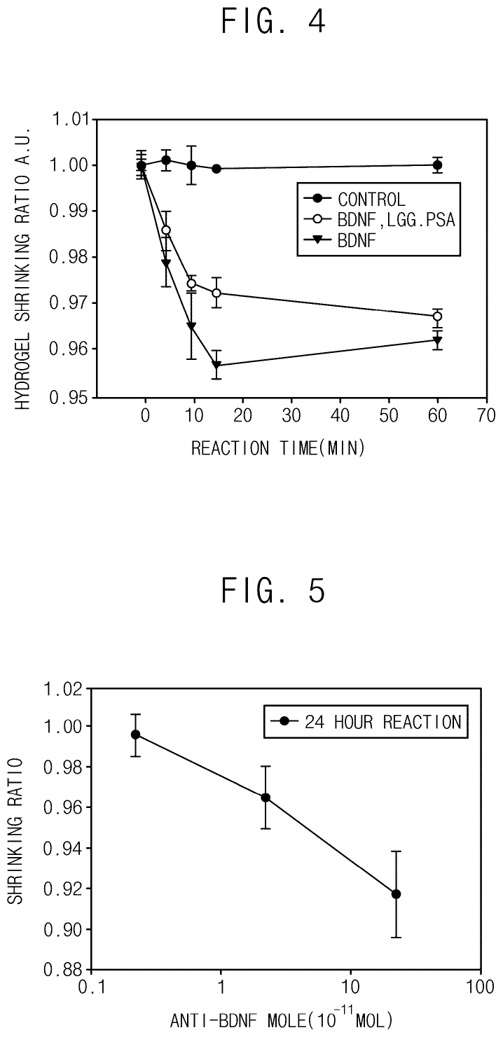

3 μL of BDNF was added to 297 μL of PBS and tapped to prepare 300 μL of BDNF antigen solution with a concentration of 111 nM. BDNF-sensitive hydrogel was added to the above antigen solution and stirred at 100 rpm at room temperature. At this time, the reaction was carried out for different times of 5 minutes, 10 minutes, 15 minutes, and 60 minutes, and the pitch size of the hydrogel was measured using an optical microscope. The result graph is shown in FIG. 4.

Compared to the untreated control group, it can be confirmed that the volume of the hydrogel was significantly reduced by BDNF.

### <Comparative Example 1> Selectivity Measurement of BDNF Detection in a Competitive Environment

The hydrogel pitch size was measured in the same manner as in Example 1, except that 1 μL each of BDNF, IgG (platelet-associated IgG), and PSA (prostate specific antigen) was added to 297 μL of PBS and tapped to prepare 300 μL of a mixed solution of BDNF, IgG, and PSA antigens, with each antigen having a concentration of 111 nM. The resulting graph is shown in FIG. 4.

The hydrogel volume change rate when BDNF alone was used according to Example 1 and the hydrogel volume change rate when a mixed solution of BDNF, IgG, and PSA was used according to Comparative Example 1 were observed at similar levels. That is, this means that the target analyte maintains equivalent selectivity even in competition with other antigens, and the excellent selectivity of the sensing system according to the present invention could be confirmed through the above results.

According to the present example embodiments, by quantifying the volume change rate of the hydrogel through the Moiré signal, the presence and quantitative detection of target analyte may be easily implemented.

In addition, by utilizing the transparent properties of the hydrogel, the target analyte is detected through moiré pattern analysis, unlike conventional optical analysis methods that utilize fluorescent substances, so there is no need to add labeling substances, and detection may be performed in a simple manner.

In addition, by involving compounds with biochemical functional groups in the polymer chain polymerization process, it provides the advantage of being able to detect various types of markers.

In addition, by amplifying the detection signal of the target analyte through changes in the moiré pattern, detection of extremely small amounts of the target analyte is possible, and the amplification effect of the detection signal may be easily controlled by controlling the concentration of the target analyte-specific probe.

Although the exemplary embodiments of the present invention have been described, it is understood that the present invention should not be limited to these exemplary embodiments but various changes and modifications can be made by one ordinary skilled in the art within the spirit and scope of the present invention as hereinafter claimed.

What is claimed is:

1. A method for detecting a target analyte using hydrogel, the method comprising:
preparing first and second polymer hydrogels having first and second patterns respectively, the first polymer hydrogel having a target analyte-specific probe coupled thereto and having target analyte sensitivity;
contacting a target analyte with the first polymer hydrogel to induce a volume change of the hydrogel; and
obtaining a change in a moiré pattern generated by overlapping the first and second polymer hydrogels with each other from an image monitoring device.

2. The method of claim 1, wherein the target analyte-specific probe is cross-linked with a polymer chain inside the first polymer hydrogel and is fixed to a hydrogel surface.

3. The method of claim 1, wherein the second pattern is a reference pattern that overlaps the first pattern to form the moiré pattern.

4. The method of claim 1, further comprising:
quantifying a change in the moiré pattern.

5. The method of claim 1, wherein the change of the moiré pattern is induced by a probe immobilized in a polymer hydrogel sensitive to the target analyte and a shrinkage of the hydrogel by a specific binding of the target analyte.

6. The method of claim 1, wherein each of the first and second patterns is in a form of a parallel grid with a regular interval of 5 nm to 100 nm.

7. The method of claim 1, wherein the probe is at least one selected from a group consisting of an aptamer, a peptide, an enzyme, a hormone receptor, an antibody, an antigen, and a cell.

8. The method of claim 1, wherein a polymer is at least one selected from a group consisting of polyethylene glycol (PEG), polyvinyl alcohol (PVA), polyvinyl pyrrolidone (PVP), polyacrylamide, polyacrylic acid and copolymers thereof, alginate, agarose, cellulose, gelatin, collagen, hyaluronic acid and chitosan.

9. The method of claim 1, wherein the target analyte-specific prove has an acrylate functional group introduced.

10. A biosensing device comprising:
a reaction part (30) comprising a first polymer hydrogel having a first pattern, the first polymer hydrogel having a target analyte-specific probe coupled thereto and having target analyte sensitivity;
a light source part (10) providing a light to the reaction part (30);
a reference pattern part (20) comprising a second polymer hydrogel having a second pattern, and disposed in an optical path between the reaction part (30) and the light source part (10), wherein the second pattern is projected onto the first pattern by the light provided by the light source part (10) to form a moiré pattern;
an imaging part (40) configured to make an image of the first pattern and the second pattern projected onto the first pattern; and
a processing part (50) configured to analyze target analyte information based on the moiré pattern obtained by the imaging part (40), wherein the moiré pattern changes as the target analyte binds to the target analyte-specific probe.

11. The biosensing device of claim 10, wherein the target analyte-specific probe is cross-linked with a polymer chain inside the first polymer hydrogel and fixed to a hydrogel surface.

12. The biosensing device of claim 10, wherein the second pattern is a reference pattern that overlaps the first pattern to form the moiré pattern.

13. The biosensing device of claim 10, wherein a change of the moiré pattern is induced by a probe immobilized in a polymer hydrogel sensitive to the target analyte and a shrinkage of the hydrogel by a specific binding of the target analyte.

14. The biosensing device of claim 10, wherein the probe is at least one selected from a group consisting of an aptamer, a peptide, an enzyme, a hormone receptor, an antibody, an antigen, and a cell.

15. The biosensing device of claim 10, wherein a polymer is at least one selected from a group consisting of polyethylene glycol (PEG), polyvinyl alcohol (PVA), polyvinyl pyrrolidone (PVP), polyacrylamide, polyacrylic acid and copolymers thereof, alginate, agarose, cellulose, gelatin, collagen, hyaluronic acid and chitosan.
